(19) United States
(12) Patent Application Publication
(10) Pub. No.: US 2011/0124106 A1
(43) Pub. Date: May 26, 2011

(54) SEPARATION DEVICE

(75) Inventors: Gunnar Froman, Uppsala (SE); Henrik Ostlin, Uppsala (SE)

(73) Assignee: GE HEALTHCARE BIO-SCIENCES AB, UPPSALA (SE)

(21) Appl. No.: 13/055,800

(22) PCT Filed: Jul. 20, 2009
(86) PCT No.: PCT/SE2009/050909

§ 371 Date: Jan. 25, 2011

(30) Foreign Application Priority Data
Jul. 31, 2008 (SE) 0801746-9

(51) Int. Cl. C12N 5/071; B01D 35/28

(52) U.S. Cl. 435/379; 210/416.1; 210/350

(57) ABSTRACT

A separation device for separating a wanted end product from a liquid sample comprises a container (2) having a first end (5) and a second end (7), the first end having a central orifice (6), a plunger (3) slideably disposed in the container (2) to define a variable liquid receiving chamber between the plunger (3) and the orifice (6), and a permeable partition member (9) mounted to the plunger (3) in a spaced relationship thereto to define a compartment (12) between the partition member (9) and the plunger (3) for receiving liquid density gradient medium (13), wherein liquid may be drawn into the container (2) and expelled therefrom, respectively, through the orifice (6) by movement of the plunger (3) relative to the container (2). A method for separating a wanted end product from a liquid sample is also disclosed.

### CROSS-REFERENCE TO RELATED APPLICATIONS

This application is a filing under 35 U.S.C. §371 and claims priority to international patent application number PCT/SE2009/050909 filed Jul. 20, 2009, published on Feb. 4, 2010 as WO 2010/014033, which claims priority to application number 0801746-9 filed in Sweden on Jul. 31, 2008.

### FIELD OF THE INVENTION

The present invention relates to a separation device adapted for separation of a wanted end product from a sample by centrifugation, and to a method for separation of a wanted end product from a sample using the separation device.

### BACKGROUND OF THE INVENTION

The separation of cell containing samples, for example blood, into different fractions by using centrifugation and a density gradient medium has been practised for some time. The principle used is to provide for example a blood sample together with a density gradient medium in a tube and then put the tube into a centrifuge. The density gradient medium is suitably chosen such that after centrifugation red blood cells are collected at the bottom of the tube, below the density gradient medium, and the wanted fraction, for example mono nuclear cells, MNCs, will stay at the top of the density gradient medium. The plasma will also be separated and stay above the MNCs. In order to collect the MNCs a pipette is normally used. Typically, the pipette is manually lowered into the tube such that the open end of the pipette is provided in the MNC band. Thereafter the MNCs are manually drawn up through the pipette. This is a tricky process since only MNCs are wanted. The amount of density gradient medium and plasma should be minimised. Such a manual process using centrifugation and a density gradient medium is for example described in Boyum, A. Isolation of mononuclear cells and granulocytes from human blood. Scand. J. Clin. Lab. Invest. 21, Suppl 97 (Paper IV), 77-89, 1968.

A problem with this method is as described above that the manual handling of the pipette when collecting the MNCs is difficult. The yield and purity of the end product will differ due to variations in the collection.

Another problem is related to the sample application. The sample needs to be applied very carefully on top of the density gradient medium in order not to be mixed with the density gradient medium before centrifugation.

### SUMMARY OF THE INVENTION

One object of the invention is to provide a separation device that is easy to use, including easy sample application and easy withdrawal of sample, where the wanted end product easily can be retrieved as pure as possible.

Another object of the invention is to provide a separation device which has a simple design and construction and is suitable for mass production.

These objects are achieved with a separation device according to claim 1 and with a method according to claim 11. With this device and method it is easy to apply the sample and easy to retrieve the wanted end product.

Suitable embodiments are described in the dependent claims.

### BRIEF DESCRIPTION OF THE DRAWINGS

FIG. 1 is a schematic cross-sectional view of a syringe device according to a first embodiment of the invention.

FIGS. 2A and 2B are schematic illustrations of different steps when using the syringe device in FIG. 1 for separation of a sample.

FIG. 3 is a schematic view of a syringe device according to a second embodiment of the invention.

FIG. 4 is a schematic partial view of an embodiment of a collapsible partition member.

FIG. 5 is a schematic illustration of different steps when using the syringe device in FIG. 3 for separation of a sample.

FIG. 6 is a schematic illustration of different steps when using the syringe device in FIG. 3 for separation of a sample by means of a linear gradient of density gradient medium.

### DETAILED DESCRIPTION OF THE INVENTION

According to the invention, a syringe device and a method for the separation of a wanted end product from a sample is provided.

The sample could be for example a body fluid such as blood or bone marrow, a body tissue, such as adipose tissue or a sample containing cell cultures or cell clusters or cell fragments such as organelles. The wanted end product could be cells of different kinds, such as for example stem cells, mononuclear cells (=MNCs), hematopoetic cells and progenitor cells or cell fragments/organelles such as for example mitochondria, golgie, endoplasmic reticulum and cell nuclei.

A density gradient medium (density separation medium) is provided inside the syringe before sample is applied and the syringe is centrifuged. The term “density gradient medium” is to be interpreted in a broad sense herein. While the density gradient medium usually is a medium which may form a density gradient upon centrifugation or sedimentation, it may also be a medium which does not form a density gradient but merely has a different density than the sample medium and forms a step gradient with the sample medium. Density gradient media used for this type of separation, such as for example FICOLL™, PERCOLL™, sucrose, inorganic salt, e.g. caesium chloride, are well known in the art. The density of the medium should be chosen such that at least one fraction of the body fluid will be separated and positioned below the density gradient medium after centrifugation. In case, for example, blood is separated, the red blood cells should preferably be separated and positioned at the bottom of the device under the density gradient medium after centrifugation.

Some density gradient media, like FICOLL™, for example, efficiently aggregate red blood cells at room temperature. When on centrifugation with such a medium, cells in a blood sample sediment towards and come in contact with the blood/density gradient medium interface, the red blood cells start to aggregate which increases the rate of sedimentation of the red cells. The red cells therefore rapidly collect as a pellet at the bottom of the syringe, where they are well separated from lymphocytes. Granulocytes will also sediment to the bottom of the density gradient medium layer, facilitated by the increase in their densities caused by contact with the slightly hypertonic gradient density medium. Thus, on completion of centrifugation, both granulocytes and red blood cells will be found at the bottom of the syringe, beneath the gradient density medium. Lymphocytes, monocytes and platelets, on the other hand, are not dense enough to penetrate into the gradient density medium layer. These cells will therefore collect as a concentrated band at the interface between the original blood sample and the gradient density medium layer.

Alternatively, two or more different density gradient media may be used. If two density gradient media are used and blood is the sample that should be separated, the density gradient medium with lowest density can preferably be of such composition that the red blood cells are not caused to aggregate. Examples of such density gradient media are PERCOLL™ or sucrose. This is to prevent red blood cells from possibly enclosing wanted cells during the aggregation process and thereby decrease yield of the wanted end product. The density gradient medium with higher density can, however, be of such composition that aggregation of the red blood cells is induced, such as e.g. FICOLL™ as mentioned above.

As still another alternative, a linear gradient produced by mixture of two different densities of a density gradient medium (or, optionally, two different density media) may be used, as will be described in more detail below.

A characteristic feature of the syringe device of the invention is a partition member attached to the syringe plunger and spaced a predetermined distance thereto to define a gradient density medium compartment between the plunger and the partition member. The partition member should, on the one hand, prevent mixing of the gradient density medium with sample fluid applied on top of the partition member but, on the other hand, be permeable to permit passage of liquid and usually also of sample components, such as e.g. cells, therethrough by the application of a force, such as by centrifugation or other forced movement of the partition member relative to the liquid.

The partition member may, for example, be passive like a filter, a grid, etc, preferably of capillary type, or active, such as e.g. a plate with densely packed microvalves.

In one embodiment, the partition member is rigidly fixed to the plunger. In this case, if the sample is blood, for example, on completed centrifugation the desired MNC band will be on top of the density gradient medium slightly above the partition member (i.e. at the interface between sample and density gradient medium).

In another (currently preferred) embodiment, the partition member is “collapsible” before or during centrifugation to permit displacement of the partition member towards the syringe plunger. Thereby, the distance between the partition member and the MNC band will be sufficiently increased to facilitate the harvesting or collection of the MNC band.

Such a collapsible partition member, e.g. a capillary filter, may be accomplished in various ways. For instance, the attachment of the partition member to the syringe plunger may be designed to cause displacement of the partition member towards the plunger when affected by a sufficient centrifugal force during centrifugation. Alternatively, the attachment of the partition member may be designed to permit such displacement by, e.g., manual actuation of the plunger or partition member before centrifugation. In still another alternative, the attachment of the partition member may be designed to permit the plunger to be displaced towards the partition member.

Collapsibility by manual actuation may be accomplished, for example, by the plunger and the partition member being attached to each other through two (or more) telescoping sliding or threadedly engaged cylinders or the like, whereby one cylinder may be partly or wholly pushed (optionally by centrifugal force) or screwed, respectively, into the other. Another example of collapsible attachment structure is a bellows type member which may be compressed on centrifugation and kept in the collapsed state by a suitable latch or friction means, for instance. In still another example, the partition member is mounted to the upper end of a shaft or bar member which sealingly and slidingly extends through the center of the syringe plunger, so that axial movement of the shaft or bar varies the distance between the plunger and the partition member.

Embodiments of collapsible partition members will be described in more detail below.

The desired cell band or bands may be removed from the syringe device and collected in various ways. Suitable collection means include, for example, flexible bags, vials, tubes, or other containers or receptacles.

In one embodiment, collection of waste liquid (the plasma) as well as of the desired MNC band(s) takes place through flexible containers attached to the syringe inlet/outlet. After the sample has been introduced into the syringe, and optionally after centrifugation to cause cell banding, a first flexible container is attached to the syringe. The supernatant plasma fraction may then be displaced into the container by, for example, centrifugation at a higher speed or by manual or automatic displacement of the syringe plunger towards the syringe inlet/outlet in a dedicated holder or similar device. A second (smaller) container is then attached to the syringe to collect the desired MNC band (or bands) by manual or automatic operation of the syringe in the dedicated holder. The collected MNC band is then ready for further processing.

Alternatively, the syringe contents may be displaced via an applied conduit, such as a tubing, into a number of fraction collection tubes or the like. The displacement through the tubing is then monitored visually or by a detection means, e.g. a photocell, so that various separated fractions may be collected in respective collection tubes. Such a process may, of course, also be automated.

Optionally, an initial step of depletion of unwanted cells may precede the density separation. For example, the sample may be pre-incubated with beads containing immobilized affinity ligands specific to the unwanted cells.

An automated or semi-automated separation procedure according to the present invention including such pre-treatment of the sample may comprise the following steps:
Sample is pre-incubated with beads.
A syringe device containing density gradient medium is placed in a dedicated holder and sample is applied by actuation of the holder, such as by pushing a button.
The syringe device is capped and placed in a centrifuge, and centrifuged to separate the sample components.
The syringe device is then again placed in the dedicated holder and a tubing is connected, whereupon cell fractionation is started, such as by pushing a button.

In case a linear density gradient is to be used, such a gradient may be formed by placing an empty syringe device in the holder and applying a varying mixture of high and low density medium, and then applying the sample.

Embodiments of the present invention will now be described in more detail with reference to the accompanying drawings.

### First Embodiment

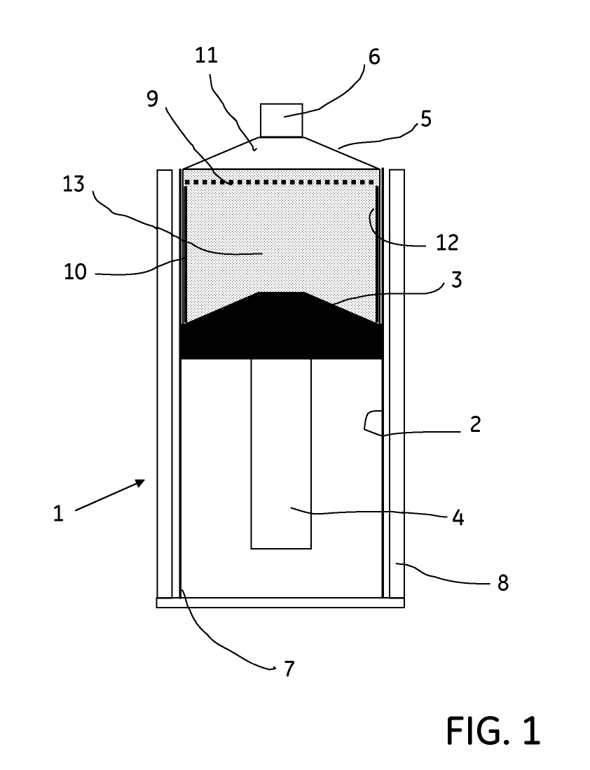

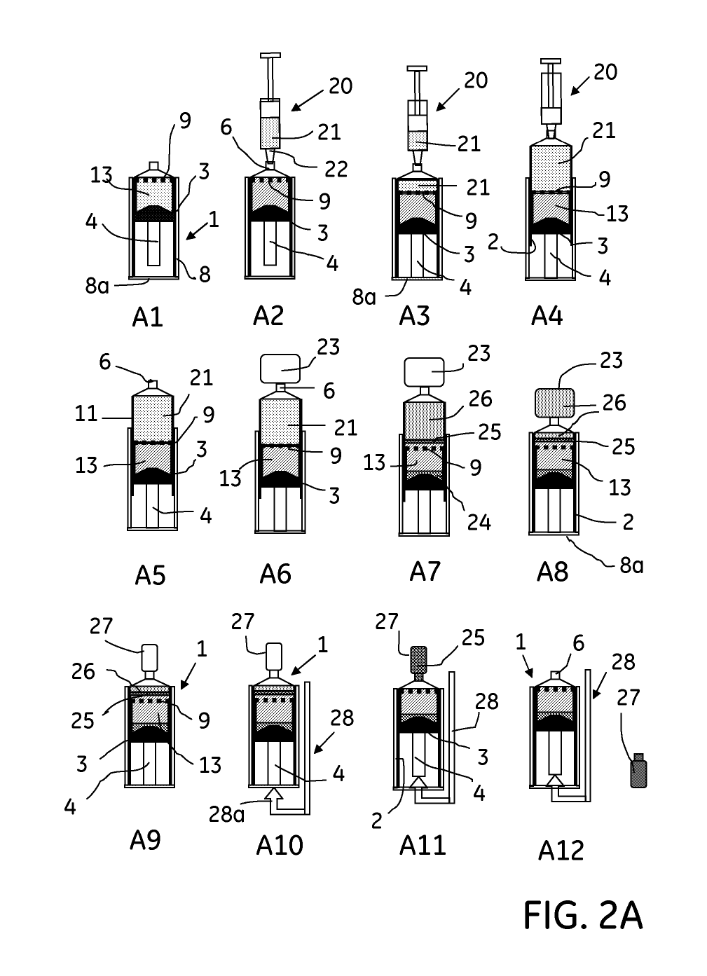

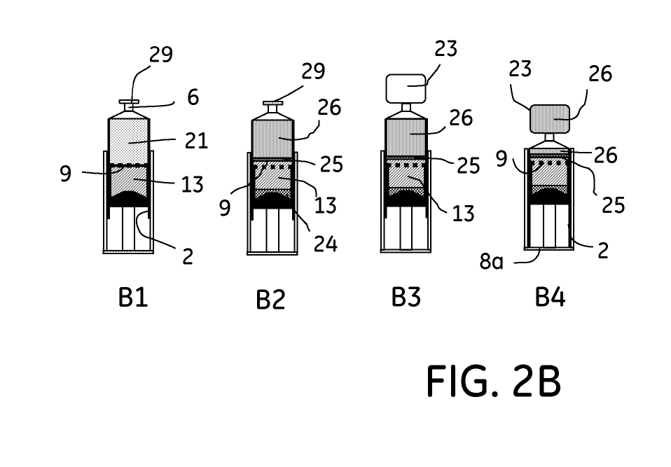

An embodiment of the syringe device of the present invention is illustrated in FIGS. 1 and 2A, 2B. With specific reference to FIG. 1, the syringe device, generally designated by reference numeral 1, includes a syringe cylinder 2 in which a plunger including a piston plug 3 with a piston rod 4 is slideably mounted. The syringe cylinder 2 has a tapering top end 5 (here a frustrated cone) with a sample inlet/outlet 6, and an open bottom end 7 (“top” and “bottom” referring to the position of the syringe device 1 on the drawing). The syringe cylinder 2 is slideably mounted in a supporting cylinder or bucket 8. A partition member 9 in the form of a filter or grid, for example, in the following for simplicity referred to as filter 9, is rigidly attached to the piston plug 3 spaced thereto, e.g. by a cylindrical member 10. In the Figure, the filter 9 is shown to be at its top position in the syringe cylinder 2 adjacent to the cone-shaped top end 5, and with the bottom end of the piston rod 4 spaced from the bottom of the supporting bucket 8. The filter 9 divides the volume between the piston plug 3 and the inlet/outlet 6 into a sample compartment 11 above the filter 9, and a density gradient medium compartment 12 below the filter 9. As is readily seen, the volume of the density gradient medium compartment 12 is constant, whereas the volume of the sample compartment 11 varies depending on the position of the piston plug 3 in the cylinder 2. In the Figure, the compartment 12 is filled with a liquid density gradient medium 13. The component parts of the syringe device 1 are made from a suitable material known to a person skilled in the art.

The syringe device 1 is designed to be placed in the rotor of a centrifuge, either directly or through a suitable adapter.

A method of using the syringe device in FIG. 1 will now be described with reference to FIGS. 2A and 2B.

In FIG. 2A, the different method steps are illustrated by subfigures A1 to A12.

A1: The syringe device 1 is ready for use with the filter 9 in its top position as shown in FIG. 1, i.e. with the piston rod 4 at a distance from the bottom end 8a of the supporting bucket 8, and with the compartment 12 filled with density gradient medium 13, e.g. FICOLL™, up to the top surface of filter 9.

A2: A sample applicator 20 with sample 21, e.g. blood, is connected via a tip portion 22 to the inlet/outlet 6 of the syringe device 1. Optionally, the sample has first been subjected to a pre-incubation with beads containing immobilized specific affinity ligands for depletion of unwanted cells.

A3: Sample 21 is applied to the syringe device 1 by actuation of the sample applicator 20. Initially, the introduced sample 21 forces the piston plug 3 downwards until the piston rod 4 contacts the bottom 8a of the supporting bucket 8. During the whole sample application process, the filter 9 keeps the sample separated from the density gradient medium 13.

A4: Continued application of sample 21 then forces the syringe cylinder 2 to be displaced upwards relative to the piston plug 3 with the attached filter 9.

A5: When all sample 21 (or the desired amount of sample) has been introduced into the sample compartment 11, the sample applicator 20 is removed.

A6: A flexible waste container 23 is attached to the syringe inlet/outlet 6 to serve as a waste compartment. Preferably, the inlet of the flexible container 23 is constrained in the sense that a predetermined force or pressure is necessary to permit the entry of fluid into the container.

A7: The syringe assembly is then placed in a centrifuge and centrifugation is started. During centrifugation at a selected first speed, the cells in the sample 21 are forced through the filter 9 and separated and banded in the density gradient medium 13. When the sample is blood, for example, the red cells (which have a specific gravity higher than the selected density gradient medium) pass through the density gradient medium 13 and are consolidated in a layer 24 at the bottom of the density gradient medium compartment 12, whereas the wanted fraction containing MNCs (which have a specific gravity less than the density gradient medium) is concentrated in a band 25 on top of the density gradient medium 13 above the filter 9 at the interface between the plasma 26 and the density gradient medium.

A8: The centrifugal force is then increased by centrifugation at a second, higher speed which forces the syringe cylinder 2 to move downwards to eventually contact the bottom 8a of supporting bucket 8, thereby displacing the plasma 26 into the flexible container 23.

Alternatively, the separation step in A7 and the plasma displacement step in A8 may be combined and done simultaneously by controlling the centrifugation force to fine-tune the speed of cells sedimentation and displacement of the plasma.

As still another alternative, the displacement of the plasma may be done manually or automatically in a dedicated holder or apparatus similar to, or the same as that outlined with reference to subfigures A9 to A12 below.

A9: The flexible container 23 with plasma 26 is then removed, and replaced by a (small) sample container 27.

A10: In the shown embodiment, the syringe device 1 is then put in a dedicated (specially designed) holder or apparatus, schematically illustrated at 28, which has a part 28a capable of actuating the piston rod 4, either by manual operation or automatically.

A11: By actuation of the piston rod 4 in the holder 28, here simply by pressing syringe device 1 downwards in the holder, the piston plug 3 is moved upwards in the syringe cylinder 2, displacing the MNC band 25 into the sample container 27.

A12: When the displacement of the MNC band is completed, the sample container 27 containing the MNCs is removed and optionally capped.

An alternative way of performing the cell banding centrifugation and displacement centrifugation described in the steps of subfigures A6 to A8 in FIG. 2A above is illustrated in FIG. 2B by subfigures B1 to B4 (wherein the same reference designations as in FIG. 2A are used for corresponding parts).

B1: After the sample applicator 20 has been removed (subfigure A5 in FIG. 1), a stopper 29 is put on the syringe inlet/outlet 6.

B2: Cell separation centrifugation at a first speed is then performed as described above until the red blood cells 24 are consolidated at the bottom of compartment 12 and the MNC band 25 stays on top of the filter 9 at the interface between plasma 26 and density gradient medium 13.

B3: The stopper 29 is then removed and replaced by a flexible container 23.

B4: Continued centrifugation at a second speed displaces the plasma 26 into the container 23, the syringe cylinder 2 being forced downwards to contact the bottom 8a of the supporting bucket 8. Optionally, this displacement of the plasma may instead be done manually or automatically in a dedicated holder or apparatus similar to, or the same as that outlined with reference to subfigure A10 in FIG. 2A. Harvesting of the MNC band is then carried out as described with reference to subfigures A9 to A12 above.

### Second Embodiment

### Step Density Gradient Separation

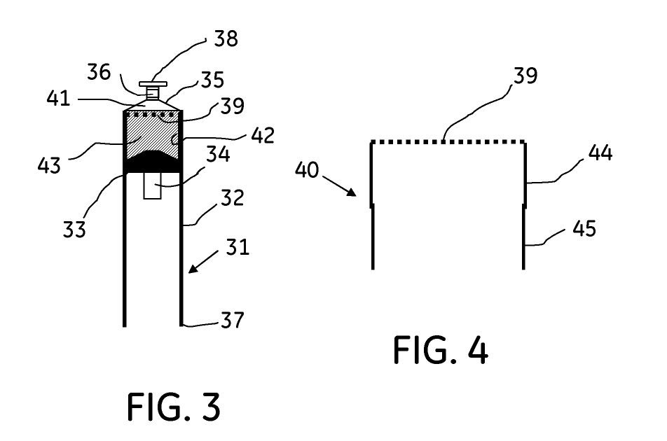

Another embodiment of the syringe device of the present invention is illustrated in FIGS. 3 to 6. With specific reference to FIG. 3, the syringe device, generally designated by the reference numeral 31, similarly to the syringe device in FIGS. 1 and 2A, 2B comprises a syringe cylinder 32 in which a plunger or piston including a piston plug 33 and a piston rod 34 is slidingly mounted. The top end 35 of the cylinder 32 tapers to a sample inlet/outlet 36, whereas the bottom end 37 of the cylinder is open. In the illustrated case, the inlet/outlet 36 is capped by a stopper 38. A partition member 39 in the form of a filter or grid, for example, in the following for simplicity referred to as filter 39, is attached to the piston plug 33 and divides the volume enclosed between the piston plug 33 and the inlet/outlet 36 into a sample compartment 41 above the filter 39 and a compartment 42 below the filter 39 for containing a density gradient medium 43.

In contrast to the embodiment in FIGS. 1 and 2A, 2B, the filter 39 in the syringe device 31 is mounted to the piston plug 33 such that the distance between the filter and the piston plug is adjustable to enable the filter 39 to be displaced relative to the piston plug 33 from an outermost position where the filter is at a fixed distance from the piston plug, to an innermost position where the filter is considerably closer to the piston plug. The mounting of the filter to the piston in order to permit such displacement or “collapse” of the filter 39 may be designed in various ways and may be made to take place in a continuous or stepwise manner, such as in a single step. The means to cause such collapse of the filter may also be designed in various ways depending on the mounting of the filter to the piston plug.

FIG. 4 schematically shows an embodiment of a filter-piston plug mounting where collapse of the filter may be caused when a force exceeding a predetermined value acts on the filter, such as e.g. the force on the filter when the syringe device 31 is centrifuged at a sufficiently high speed. In FIG. 4, the filter 39 is attached to the piston plug (not shown) by a cylindrical two-part member 40 consisting of a cylindrical filter holder 44 and a cylindrical support 45 fixed to the piston plug (not shown). The holder 44 has a slightly larger internal diameter than the external diameter of the support 45, so that collapse of the filter may be caused by overcoming the friction between the holder 44 and the support 45. In the collapsed position, the filter 39 is adjacent to or rests on the top edge of support 45.

In a variant (not shown), the holder 44 is threadedly engaged with the support 45 so that collapse of the filter may be accomplished by rotating the piston plug (via the piston rod) relative to the filter 39. Numerous other variants are conceivable to the skilled person.

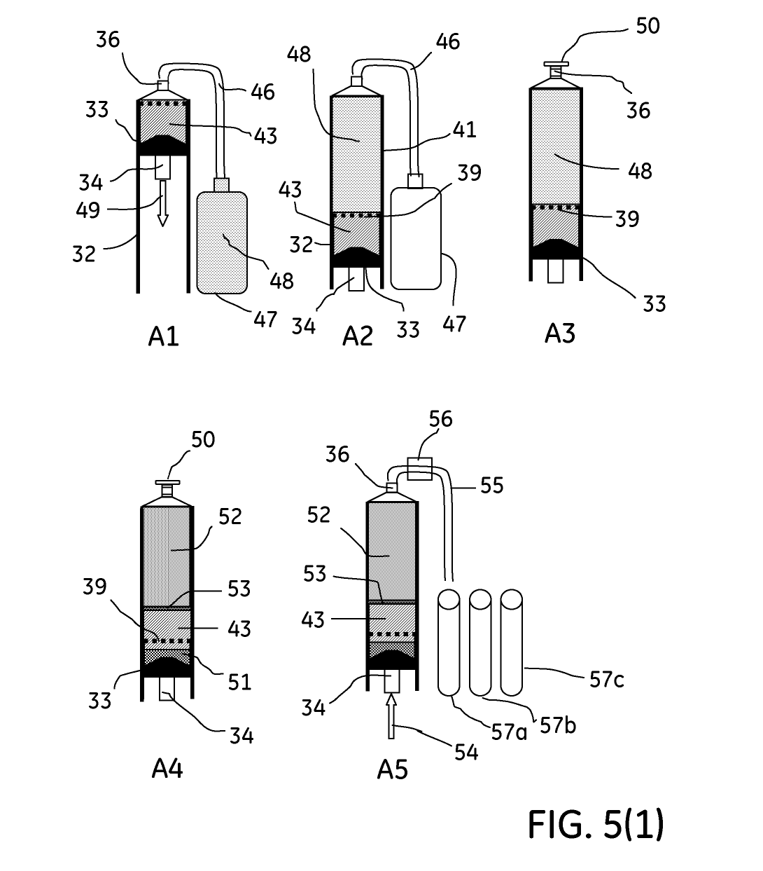

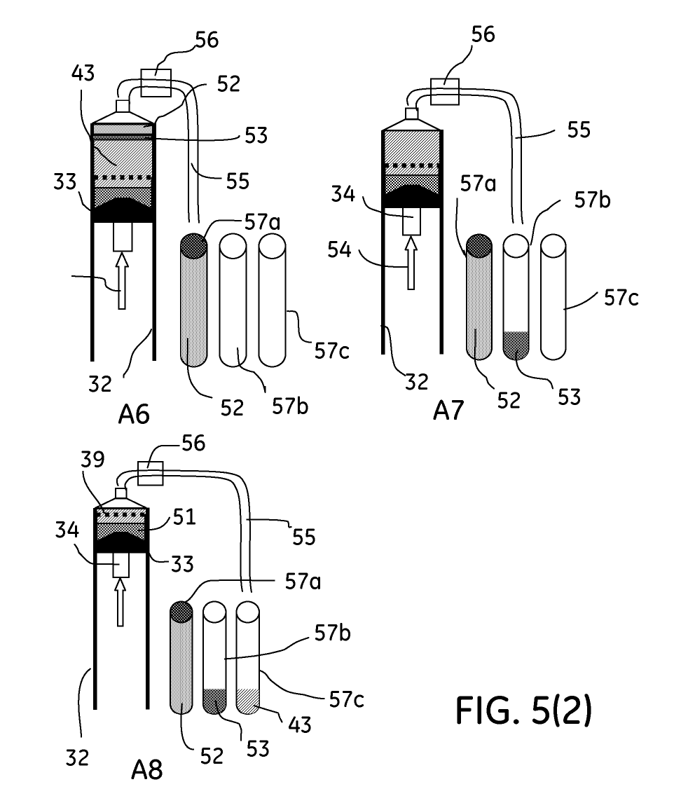

A method embodiment of using the syringe device illustrated in FIGS. 3 and 4 in a step density gradient separation process will now be described with reference to FIG. 5, where the different method steps are schematically illustrated by subfigures A1 to A8.

A1: The syringe is placed in a dedicated holder (not shown), and a tube 46 connected to a container 47 with sample 48 is attached to the syringe inlet/outlet 36. The holder is provided with actuation means (not shown) for actuating the piston rod 34 to displace the piston plug 33 vertically within the syringe cylinder 32. Such means may be manual or automated. The actuation means is then operated to initiate displacement of the piston plug downwards as indicated by arrow 49. As shown in subfigure A2 below, the filter 39 prevents mixing of the sample with the density gradient medium 43.

A2: All sample 48 has now been sucked into the expanded compartment 41 of the syringe, the filter 39 having moved together with the piston plug 33 with the density gradient medium 43 enclosed between them. In the illustrated case, the end of the piston rod 34 has reached the level of the open end of the syringe cylinder 32.

A3: The syringe device is then capped by applying a stopper 50 to the syringe inlet/outlet 36, removed from the holder device, and put in a centrifuge (not shown) and centrifuged. During the centrifugation, the filter 39 “collapses”, i.e. is displaced towards the piston plug 33 by the centrifugal force making the filter holder 44 (FIG. 4) overcome the friction and slide down the support 45 (FIG. 4), simultaneously as the sample components are separated.

A4: When the centrifugation is completed, (in the case of the sample being blood) the red blood cells are consolidated in a layer 51 on top of the piston plug 33, whereas the plasma 52 is above density gradient medium 43. The MNH cells are banded in layer 53 at the plasma/density gradient medium interface. The filter 39 is in its collapsed position close to the piston plug 33, so that the MNC cell band 53 is at a considerable distance from the filter 39.

A5: The syringe device is then again put in the dedicated holder (not shown) referred to above in connection with step A1, so that the piston rod 34 can be actuated to move upwards as indicated by arrow 54. The stopper 50 is removed, and a tubing 55 is connected to the inlet/outlet 36 of the syringe device. Optionally, an optical detector 56 is mounted on the tubing 55. A number of containers for collection of fluid expelled from syringe device, here three collector tubes 57a-57c, are provided at the other end of the tubing 55. Fractionation of the syringe contents is initiated by actuation of the piston rod 34 in the holder with the tubing 55 opening into collector tube 57a.

A6: The piston plug 33 is moved upwards, while the first fraction, i.e. the cell-free plasma 52, is collected in the first collector tube 57a. In the Figure, the MNC band 53 has almost reached the top end of the syringe cylinder 32.

A7: Further displacement of the piston plug 33 upwards in the syringe cylinder 32 expels the MNC band 53 out of the syringe device into the tubing 55. This is detected by the detector 56 (or optionally visually) and the end of tubing 55 is moved to open into the second collector tube 57b for harvest of the MNC cells 53 therein. This may be done manually, but may also be done automatically by the holder device being triggered by the detector 56.

A8: When the MNC cell band 53 has been completely expelled from the syringe device as detected by detector 56 (or visually) and collected in collector tube 57b, the end of tubing 55 is moved to the third collector tube 57c for collection of waste density gradient medium 43 which is expelled on continued upwards displacement of the piston plug 33 until the filter 39 is at its top position in syringe cylinder 32.

### Linear Gradient Separation

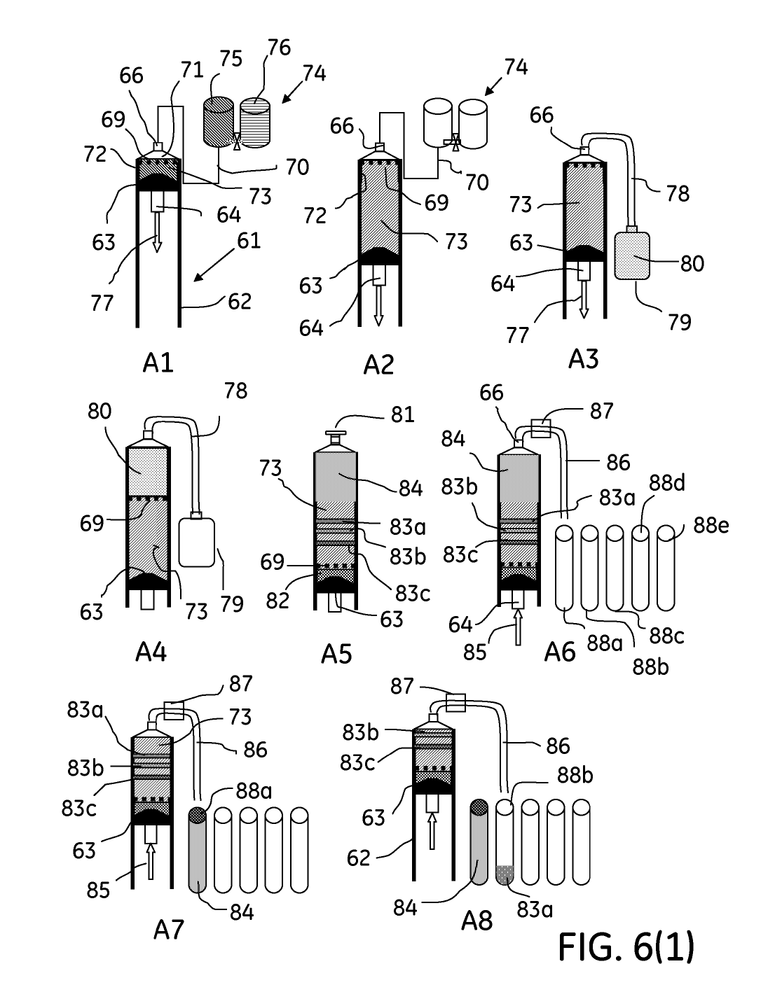

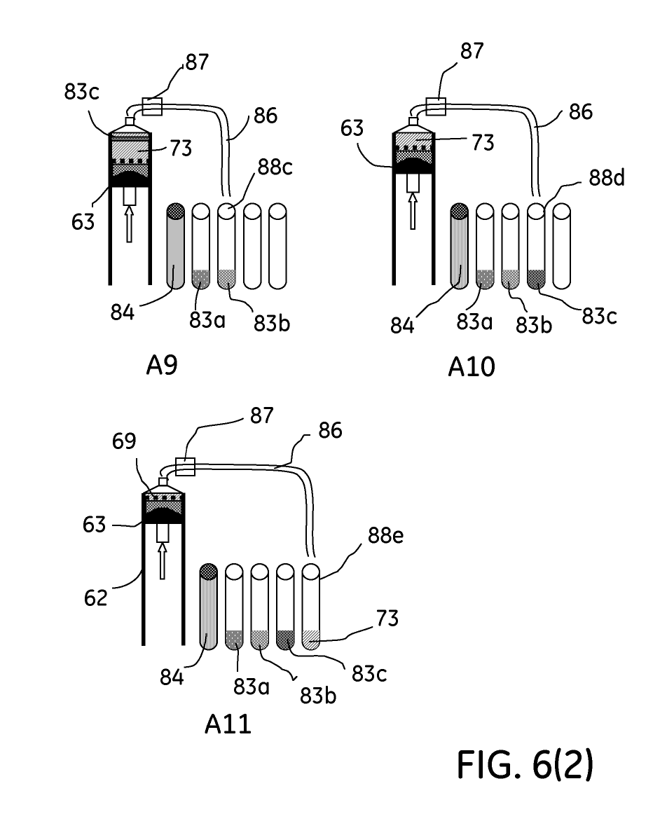

Use of a syringe device similar to that shown in FIG. 3 in a linear density gradient separation process will now be described with reference to FIG. 6, where the different method steps are schematically illustrated by subfigures A1 to A11 (identical reference numerals being used for corresponding parts). In the subfigures, the syringe device, generally designated by the reference numeral 61, is placed in the dedicated holder mentioned above or in a corresponding device (not shown).

In the different subfigures, the syringe device 61, similarly to the syringe device in FIGS. 1 to 3, comprises a syringe cylinder 62 in which a plunger or piston including a piston plug 63 and a piston rod 64 is slidingly mounted. The top end of the cylinder 62 tapers towards a sample inlet/outlet 66, whereas the bottom end of the cylinder is open. A partition member 69 in the form of a filter or grid, for example, in the following for simplicity referred to as filter 69, is attached to the piston plug 63 and divides the volume enclosed between the piston plug 63 and the inlet/outlet 66 into a sample compartment 71 above the filter 69 and a compartment 72 below the filter 69 for containing a density gradient medium.

While the filter mounting shown in FIG. 4 could per se also be used here, it is preferable to use a mounting structure capable of collapsing to a higher degree, such as may, e.g. be obtained by a telescopic assembly having more than two cylinders that slidingly fit into each other.

A1: With the syringe device 61 placed in the dedicated holder, the syringe inlet/outlet 66 is then connected via a tube 70 to a gradient mixer 74 comprising a first container 75 with a high density medium (HDM), which e.g. may be a PERCOLL™ or FICOLL™ medium with high density, and a second container 76 with a low density medium (LDH), which e.g. may be a PERCOLL™ or FICOLL™ medium with low density. Fluid from the gradient mixer 74 is introduced into the syringe device by actuating the piston rod 64 for downward movement of the piston plug 63 as indicated by arrow 77. When the piston plug 63 is moved downwards, the filter 69 in the collapsible assembly remains at the top of the syringe cylinder 62.

A2: The gradient mixer 74 has been emptied and a linear gradient of density medium 73 has been formed in the compartment 72 between the filter 69 and the piston plug 63.

A3: A tube 78 connected to a container 79 with sample 80 is then attached to the syringe inlet/outlet 66, and the piston rod 64 is again actuated for downward movement of the piston plug 63, as indicated by arrow 77. Optionally, the sample has first been subjected to a pre-incubation with beads containing immobilized specific affinity ligands for depletion of unwanted cells.

A4: All sample 80 has now been sucked into the expanded compartment 71 of the syringe, the filter 69 having moved downwards together with the piston plug 63.

A5: The syringe device is then capped with a stopper 81, removed from the holder device, put in a centrifuge (not shown) and centrifugation is started. During centrifugation, the filter 69 collapses (i.e. is displaced towards the piston plug 63) and the sample components are separated. When the centrifugation is completed, the filter 69 is at its bottom position, and (in case the sample is blood) the red blood cells are consolidated in a layer 82 on top of the piston plug 63. The MNC cells are fractionated into several bands, here three cell bands 83a-83c, below the plasma 84, all within the linear density gradient medium 73.

A6: The syringe device is then again put in the dedicated holder (not shown) referred to above, so that the piston rod 64 can be actuated to move upwards as indicated by arrow 85. The stopper 81 is removed, and a tubing 86 is connected to the inlet/outlet 66 of the syringe device. Optionally, an optical detector 87 is mounted on the tubing 86. A number of containers for collection of fluid expelled from the syringe device, here five collector tubes 88a-88e, are provided at the other end of the tubing 86. Fractionation of the syringe contents is initiated by actuation of the piston rod 64 in the holder device with the tubing 86 opening into collector tube 88a.

A7: The piston plug 63 is moved upwards, while the first fraction, i.e. the cell-free plasma 84, is collected in the first collector tube 88a together with a top portion of the density gradient medium 73.

A8: Further displacement of the piston plug 63 upwards in the syringe cylinder 62 expels the first cell band 83a out of the syringe device into the tubing 86. This is detected by the detector 87 (or optionally visually) and the end of tubing 86 is moved to open into the second collector tube 88b for harvest of the cell band 83a therein. This may be done manually, but may also be done automatically by the holder device being triggered by the detector 87.

A9: Similarly as above, the second cell band 83b is expelled from the syringe device, detected and collected in collector tube 88c by further displacement of the piston plug 63 upwards in the syringe cylinder 62.

A10: Similarly as above, the third cell band 83c is expelled from the syringe device, detected and collected in collector tube 88d by further displacement of the piston plug 63 upwards in the syringe cylinder 62.

A11: Finally, when the third cell band 83c has been expelled from the syringe device and collected in collector tube 88d, the end of tubing 86 is moved to the fifth collector tube 88e for collection of waste density gradient medium 73 expelled on continued upwards displacement of the piston plug 63 until the filter 69 is at its top position in syringe cylinder 62.

It is to be understood that the invention is not limited to the particular embodiments of the invention described above, but the scope of the invention will be established by the appended claims.

1. A separation device comprising:
a container (2) having a first end (5) and a second end (7), the first end having a central orifice (6);
a plunger (3) slideably disposed in the container (2) to define a variable liquid receiving chamber between the plunger (3) and the orifice (6); and
a permeable partition member (9) mounted to the plunger (3) in a spaced relationship thereto to define a compartment (12) between the partition member (9) and the plunger (3) for receiving liquid density gradient medium (13);
wherein liquid may be drawn into the container (2) and expelled therefrom, respectively, through the orifice (6) by movement of the plunger (3) relative to the container (2).

2. The separation device of claim 1, wherein the partition member (9) comprises a filter or a grid, especially a capillary filter.

3. The separation device of claim 1, wherein the mounting of the partition member (9) to the plunger (3) is collapsible to permit relative displacement of the partition member (9) towards the plunger (3).

4. The separation device of claim 3, wherein the partition member (3; 39) and the plunger (3) are attached to each other through telescoping means (40).

5. The separation device of claim 4, wherein the telescoping means (40) comprise cylindrical members (44, 45) forming sidewalls of the compartment (12) for receiving liquid density gradient medium (13).

6. The separation device of claim 5, wherein the cylindrical members (44, 45) are slidingly or threadedly engaged.

7. The separation device of claim 3, wherein the partition member (9) and the plunger (3) are attached to each other through bellows means.

8. The separation device of claim 3, wherein the mounting of the partition member (9) is adapted to collapse when the device is centrifuged at a predetermined rotation speed.

9. The separation device of claim 1, wherein the device is pre-filled with liquid gradient density medium (13).

10. The separation device of claim 1, wherein the device is adapted for separation of cells or cell fragments from a sample containing cells or cell fragments, especially cells from body fluids or body tissues.

11. A method for separating a wanted end product from a liquid sample with the separation device (1) of claim 1, comprising the steps of:
filling the compartment (12) between the plunger (3) and the partition member (9) with liquid density gradient medium (13) having a higher density than the sample;
applying sample (21) into the liquid receiving chamber;
subjecting the syringe device (1) to centrifugation to separate components of the sample, components (24, 25) in the sample (21) having a higher density than the density gradient medium being transferred thereto;
moving the plunger (3) towards the first end (5) to expel liquid from the syringe device (1) through the central orifice (6); and
recovering an expelled liquid fraction containing the wanted end product (25).

12. The method of claim 11, wherein expelled liquid fractions are successively collected in flexible containers (23, 27) attachable to the central orifice (6) at the first end (5) of the syringe device (1).

13. The method of claim 11, wherein a tubing (55) is attached to the central orifice (36) at the first end of the syringe device (1), and liquid fractions expelled through the tubing (55) are collected in respective different containers (57a, 57b, 57c) at the other end of the tubing (55).

14. The method of claim 13, wherein the passage of liquid fractions through the tubing (55) is monitored by detection means (56).

15. The method of claim 11, wherein the density gradient medium provides a step gradient.

16. The method of claim 11, wherein the density gradient medium provides a linear density gradient.

17. The method of claim 11, wherein the sample contains cells or cell fragments, especially cells from body fluids or body tissues.

18. The method of claim 17, wherein prior to applying the sample to the syringe device the sample is subjected to a step of removing unwanted cells from the sample.

19. The method of claim 18, wherein the step of removing unwanted cells comprises incubating the sample with beads supporting affinity ligands specific to the unwanted cells.
